(19) United States
(12) Patent Application Publication
(10) Pub. No.: US 2015/0173616 A1
(43) Pub. Date: Jun. 25, 2015

(54) SYSTEM FOR HEALTH MONITORING SENSOR PLACEMENT

(71) Applicant: Futurewei Technologies Inc., Plano, TX (US)

(72) Inventor: Adam K. Zajac, San Diego, CA (US)

(73) Assignee: Futurewei Technologies Inc., Plano, TX (US)

(21) Appl. No.: 14/139,033

(22) Filed: Dec. 23, 2013

(51) Int. Cl. A61B 5/00; H04Q 9/00

(52) U.S. Cl. CPC A61B 5/0024; H04Q 9/00

(57) ABSTRACT

Various disclosed embodiments include apparatus and systems for providing health monitoring sensor placement. An apparatus includes a sensor device, comprising one or more sensors to respectively collect sensor data, a memory configured to store the sensor data, communication logic configured to provide the sensor data to an external electronic device. The sensor device is configured to be disposed within and removably coupled to a wristband device. When the sensor device is disposed within the wristband device, the sensor device contacts the skin of a user when the wristband device is worn by the user.

### TECHNICAL FIELD

The present disclosure relates generally to sensor devices, and more particularly to a system for obtaining health monitoring data via a body worn monitor.

### BACKGROUND

The use of sensors to monitor many parameters in our daily lives has been increasing recently. Health and wellness monitoring wearable devices have been introduced in the last few years to monitor physical activities. Theses device use motion sensors such as accelerometers and/or physiological sensors such as heart rate sensors to monitor a variety of parameters such as number of steps taken, distance travelled, calories burned, etc. Some of these devices are designed to be worn or used substantially all of the time, while others may be designed to be used, or a user may prefer to use them, for specific activities only.

One drawback of the aforementioned wearable devices is that they are typically standalone devices.

### SUMMARY

According to one embodiment, there is provided a sensor device comprising one or more sensors to respectively collect sensor data, a memory configured to store the sensor data, communication logic configured to provide the sensor data to an external electronic device. The sensor device is configured to be disposed within and removably coupled to a wristband device. When the sensor device is disposed within the wristband device, the sensor device contacts the skin of a user when the wristband device is worn by the user.

In another embodiment, there is provided an article to be worn by a user. The article comprises a wristband device that at least partially covers a wrist when worn. The wristband device includes a removable portion and a sensor device configured to be disposed within and removably coupled to the wristband device via the wristband device removable portion. The sensor device comprises one or more sensors to respectively collect sensor data, a memory configured to store the sensor data, and communication logic configured to provide the sensor data to an external electronic device. When the sensor device is disposed within the wristband device, the sensor device contacts the skin of the user when the wristband device is worn by the user.

### BRIEF DESCRIPTION OF THE DRAWINGS

For a more complete understanding of the present disclosure, and the advantages thereof, reference is now made to the following descriptions taken in conjunction with the accompanying drawings, wherein like numbers designate like objects, and in which:

FIG. 1 illustrates a system for obtaining health monitoring data via a body worn monitor in accordance with disclosed embodiments;

FIGS. 2A-2D illustrate diagrams of a wristband device in accordance with disclosed embodiments;

FIG. 3 illustrates an overall block diagram of a wristband device in accordance with disclosed embodiments; and

FIG. 4 illustrates an overall block diagram of an example electronic device in accordance with disclosed embodiments.

### DETAILED DESCRIPTION

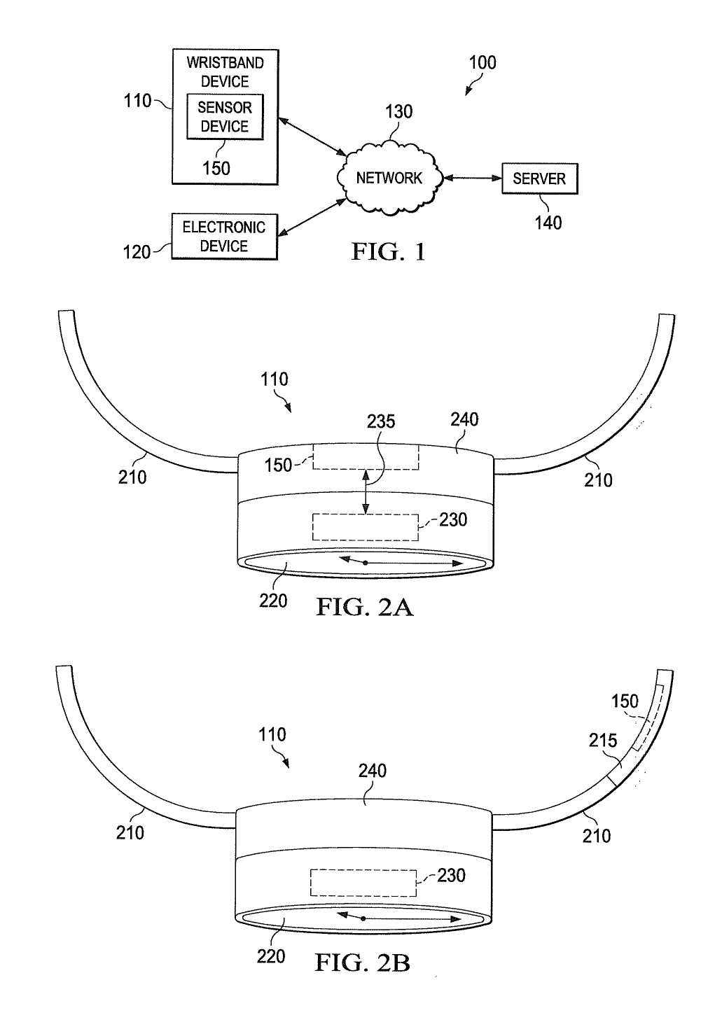

FIG. 1 is a diagram illustrating a system 100 for obtaining health monitoring data via a body worn monitor, where the body worn monitor is associated with a user's existing wrist wearable devices. The system 100 includes a wristband device 110 with a sensor device 150 disposed therein/thereon, an electronic device 120, a communications network 130 and a server 140. The wristband device 110 may be coupled to other devices via the communications network 130.

The sensor device 150 may include one or more sensors such as one or more motion sensors (e.g., accelerometers, gyroscopes, pressure sensors, etc.), one or more biometric sensors (e.g., fingerprint, face recognition, iris recognition, retina, etc.), one or more personal monitoring sensors that can sense physiological conditions (e.g., heart rate, temperature, blood glucose, etc.), or any combination thereof.

In one embodiment, the wristband device 110 may be a bracelet that includes the sensor device 150. In another embodiment, the wristband device 110 may be a watch, or in another embodiment it may be incorporated into a watch—which watch may include the traditional components of a watch. The sensor device 150 may be disposed within or attached to any part of the watch, including the watchband, the back cover of the watch, the watch face, etc. Although the device is referred to as a “wristband” device, one of skill in the art would understand that a body-worn device worn on a different part of the anatomy (e.g., arm band, leg band, headband) may be utilized to provide these features. However, for simplicity, the term “wristband” device will be used throughout this disclosure.

The electronic device 120 may be a cellular telephone, a media player with wireless communications capabilities, a handheld computer (also sometimes called a personal digital assistant), a remote controller, a global positioning system (GPS) device, a tablet computer, a desktop computer or a handheld gaming device. The electronic device 120 may be used to provide additional user interface features. In one embodiment, the electronic device 120 may also receive aggregate data collected by the wristband device 110. In one embodiment, certain calculations that are more processor intensive may take place on the electronic device 120 or on the server 140.

The wristband device 110 may also be coupled to the server 140 either directly through the network 130, or through the electronic device 120 via the network 130, to which the wristband device 110 is coupled.

The communications network 130 may be implemented by any medium or mechanism that provides for the exchange of data between various computing, sensor or communication devices. Examples of such a network include a Local Area Network (LAN), Wide Area Network (WAN), Ethernet or the Internet, or one or more terrestrial, satellite, or wireless links. The communications network 130 may include a combination of networks such as those described. Though not shown specifically in FIG. 1, the sensor device 150/wristband device 110 may communicate via wired or wireless (e.g., Bluetooth, Wi-Fi) communication links directly with the electronic device 120 (e.g., not through the network 130).

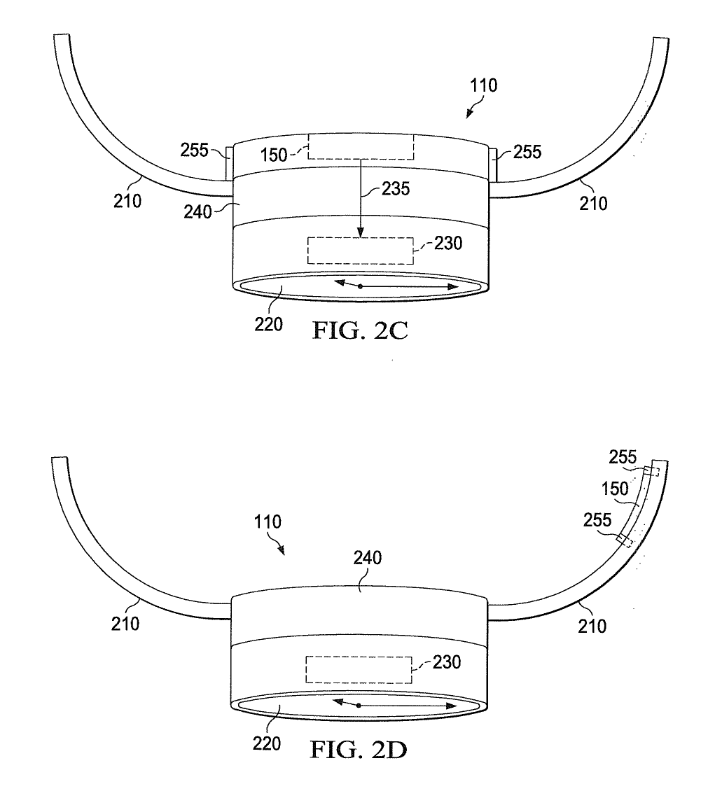

FIG. 2A illustrates a diagram of one embodiment of the wristband device 110. In the illustrated embodiment, the wristband device 110 is configured as a watch. The watch 110 includes the traditional components of a watch such as a watchband 210, a display 220 such as a watch dial or a digital display, a power source 230, and a back cover 240. The watchband 210 may include a strap, links or any combination thereof, or other configuration. The power source 230 may be associated with kinetic quartz movement, solar quartz movement, or quartz movement that includes a battery.

In the illustrated embodiment, the back cover 240 is removable, replaceable and/or configured such that the sensor device 150 is embedded within to the back cover 240, thereby providing a user with the ability to “retrofit” his or her existing watch by replacing the original back cover with a cover that includes the sensor device 150 thereby providing health monitoring functionality. A surface of the sensor device 150 may be substantially flush with a surface of the back cover 240 or may protrude slightly from a surface of the back cover 240 such that when the user secures the watch to their wrist, the sensor device 150 (and perhaps a portion of the back cover 240) contacts the user's skin. The sensor device 150 may thereafter perform its measurement function and generate sensor data.

In one embodiment, the sensor device 150 may include its own power source, such as a battery. Alternatively, or in addition, the power source 230 of the watch 110 may supply power to the sensor device 150 via a power supply interface 235 configured to provide electrical contact between the power source 230 and the sensor device 150.

FIG. 2B illustrates a diagram of another embodiment of the wristband device 110. In the illustrated embodiment, the wristband device 110 is similarly configured as a watch. The watch 110 includes the watchband 210 having a plurality of links, the display 220, the power source 230, the back cover 240, and further includes a detachable link 215 forming part of the watchband 210.

The detachable link 215 is removable, replaceable, and configured such that the sensor device 150 is embedded within the detachable link 215, thereby providing a user with the ability to retrofit his or her existing watch by replacing one of the original links with the detachable link 215 that includes the sensor device 150 thereby providing health monitoring functionality. A surface of the sensor device 150 may be substantially flush with a surface of the detachable link 215 or may protrude slightly from a surface of the detachable link 215 such that when the user secures the watch to their wrist, the sensor device 150 (and perhaps a portion of the detachable link 215) contacts the user's skin. The sensor device 150 may thereafter perform its measurement function and generate sensor data.

In this embodiment, the sensor device 150 may include its own power source, such as a battery, or alternatively (or in addition to) the power source 230 may supply power to the sensor device 150 via a power supply interface (not shown).

FIG. 2C illustrates a diagram of yet another embodiment of the wristband device 110. In the illustrated embodiment, the wristband device 110 is again configured as a watch. The watch 110 includes the watchband 210, the display 220, the power source 230, and the back cover 240. A sensor mounting device 255 is configured to couple the sensor device 150 to the back cover 240. The sensor mounting device 255 may include a snap-on attachment, an adhesive layer, fastener material such as hook and loop, and/or other mounting mechanisms.

In this illustrated embodiment, the sensor mounting device 255 functions to enable the sensor device 150 to be removably coupled to the back cover 240, thereby providing a user with the ability to retrofit his or her existing watch to provide health monitoring functionality. A surface of the sensor device 150 may be substantially flush with a surface of the sensor mounting device 255 or may protrude slightly from a surface of the sensor mounting device 255 such that when the user secures the watch to their wrist, the sensor device 150 (and perhaps a portion of the mounting device 255) contacts the user's skin. The sensor device 150 may thereafter perform its measurement function and generate sensor data.

In this embodiment, the sensor device 150 may be powered in a similar manner as described above in relation to FIG. 2A.

FIG. 2D illustrates a diagram of still another embodiment of the wristband device 110. In the illustrated embodiment, the wristband device 110 is configured as a watch. The watch 110 includes the watchband 210, the display 220, the power source 230, and the back cover 240. In the illustrated embodiment, the sensor mounting device 255 is configured to couple the sensor device 150 to the watchband 210.

In this embodiment, the sensor mounting device 255 functions to enable the sensor device 150 to be removably coupled to the watchband 210, thereby providing a user with the ability to retrofit his or her existing watch to provide health monitoring functionality. A surface of the sensor device 150 may be substantially flush with a surface of the sensor mounting device 255 or may protrude slightly from a surface of the sensor mounting device 255 such that when the user secures the watch to their wrist, the sensor device 150 (and perhaps a portion of the sensor mounting device 255) contacts the user's skin. The sensor device 150 may thereafter perform its measurement function and generate sensor data.

In this embodiment, the sensor device 150 may be powered in a similar manner as described above in relation to FIG. 2B.

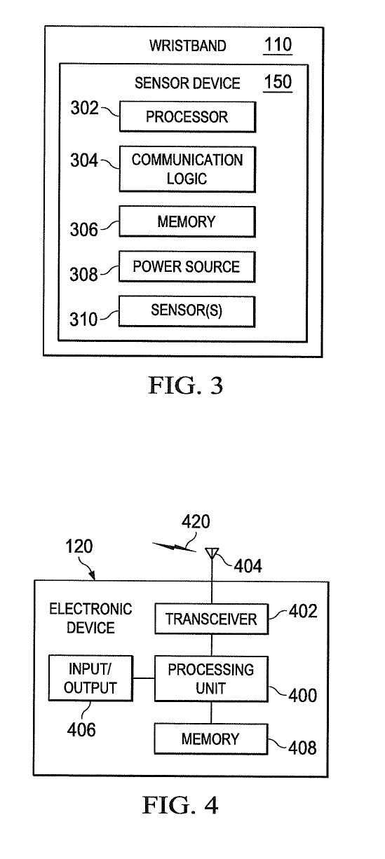

Now turning to FIG. 3, there is illustrated an overall block diagram of the wristband device 110. As illustrated, the sensor device 150 includes a processor 302, communication logic 304, memory 306, a power source 308 and one or more sensors 310. One or more other components, devices, or functionalities may be included, if desired (not shown). It will be understood that the sensor device 150 may include fewer or more of the foregoing described elements.

The processor 302 may be a general purpose, special purpose or digital signal processor, and/or may include a plurality of processors or combination of such processors. The processor 302 may include functionality to perform signal coding, data processing, power control, input/output processing, and/or any other functionality enabling the sensor device 150 to operate in the system 100 for its intended purpose.

The communication logic 304 facilitates communication between the sensor device 150 and external systems, such as the electronic device 110, the server 140 and/or the communications network. The communication logic 304 may include multiple communication mechanisms. For example, the communication logic 304 may include a bus or a network port, an RF cellular network transceiver with antenna, a Bluetooth transceiver and/or a Wi-Fi transceiver, etc. Other communications connection mechanisms may be used as well.

The memory 306 is operable for storing and retrieving data, including software and/or firmware instructions. Any suitable type of memory storage device may be included, such as random-access memory (RAM), read-only memory (ROM), and the like.

The power source 308 is operable for providing power to the sensor device 150. The power source 308 may include a battery, a capacitor, or other charge storing mechanisms.

The one or more sensors 310 are configured to collect sensor data. The one or more sensors may include one or more motion sensors (e.g., accelerometers, gyroscopes, pressure sensors, etc.), one or more biometric sensors (e.g., fingerprint, face recognition, iris recognition, retina, etc.), one or more personal monitoring sensors that can sense physiological conditions (e.g., heart rate, temperature, blood glucose, etc.), or any combination thereof.

FIG. 4 illustrates an overall block diagram of the electronic device 120. The electronic device 120 includes a processor 400, a transceiver 402, an antenna element 404, one or more input/output devices 406 (e.g., speaker/microphone, keypad, display/touchpad) and memory 408.

One or more wristband devices 110 may be wirelessly coupled to the electronic device 120 via a wireless link 420 (or wired). As will be appreciated, the electronic device 120 may include one or more other components, devices, or functionalities (not shown) as desired. It will be understood that the electronic device 120 may include fewer or more of the foregoing described elements.

The processor 400 may be a general purpose, special purpose or digital signal processor, and may be a plurality of processors or combination of such processors. The processor 400 includes functionality to perform signal coding, data processing, power control, input/output processing, and/or any other functionality enabling the electronic device 120 to operate in the system 100 for its intended purposes. The processor 400 is coupled to the transceiver 402 which is coupled to the antenna element 404. It will be understood that the processor 400 and the transceiver 402 may be separate components or integrated together. Similarly, the antenna element 404 may be a single element or a number of elements (multiple antennas or elements). The transceiver 402 is configured to transmit and receive RF signals via the antenna 404.

The processor 400 is coupled to the one or more input/output devices 406 (including ports or busses) operable for inputting/outputting user data. In addition, the processor 400 is coupled to memory 408 operable for storing and retrieving data. Any suitable type of memory storage device may be included, such as random-access memory (RAM), read-only memory (ROM), hard disk, subscriber identity module (SIM) card, a memory stick, a secure digital (SD) memory card, and the like.

In some embodiments, some or all of the functions or processes of the one or more of the devices are implemented or supported by a computer program that is formed from computer readable program code and that is embodied in a computer readable medium. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory.

It may be advantageous to set forth definitions of certain words and phrases used throughout this patent document. The terms “include” and “comprise,” as well as derivatives thereof, mean inclusion without limitation. The term “or” is inclusive, meaning and/or. The phrases “associated with” and “associated therewith,” as well as derivatives thereof, mean to include, be included within, interconnect with, contain, be contained within, connect to or with, couple to or with, be communicable with, cooperate with, interleave, juxtapose, be proximate to, be bound to or with, have, have a property of, or the like.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure, as defined by the following claims.

What is claimed is:

1. A sensor device, comprising:
one or more sensors to respectively collect sensor data;
a memory configured to store the sensor data; and
communication logic configured to provide the sensor data to an external electronic device;
wherein the sensor device is configured to be disposed within and removably coupled to a wristband device, wherein when the sensor device is disposed within the wristband device, the sensor device contacts the skin of a user when the wristband device is being worn by the user.

2. The sensor device in accordance with claim 1, further comprising:
a sensor device power source configured to provide power to the sensor device.

3. The sensor device in accordance with claim 2, further comprising:
a power source interface configured to provide electrical contact between the sensor device and a wristband device power source, the wristband device power source configured to provide power to the wristband device, wherein the sensor device is configured to receive power from one of the wristband device power source via the power source interface and the sensor device power source.

4. The sensor device in accordance with claim 1, further comprising:
a power source interface configured to provide electrical contact between the sensor device and a wristband device power source, the wristband device power source configured to provide power to the wristband device, wherein the sensor device is configured to receive power from the wristband device power source via the power source interface.

5. The sensor device in accordance with claim 1, wherein the communication logic comprises a wireless interface for wirelessly coupling the sensor device to the external electronic device.

6. The sensor device in accordance with claim 5, wherein the wireless interface is a short range wireless interface.

7. An article to be worn by a user, the article comprising:
a wristband device that at least partially covers a wrist when worn, the wristband device including a removable portion; and
a sensor device configured to be disposed within and removably coupled to the wristband device via the wristband device removable portion, wherein the sensor device comprises:
one or more sensors to respectively collect sensor data;
a memory configured to store the sensor data; and
communication logic configured to provide the sensor data to an external electronic device;
wherein when the sensor device is disposed within the wristband device, the sensor device contacts the skin of the user when the wristband device is worn by the user.

8. The article in accordance with claim 7, wherein the wristband device comprises a watch.

9. The article in accordance with claim 8, wherein the removable portion comprises a watchband link of a watchband of the watch, and wherein the sensor device is disposed within the watchband link.

10. The article in accordance with claim 8, wherein the removable portion comprises a back cover of the watch, and wherein the sensor device is disposed within the back cover of the watch.

11. The article in accordance with claim 10, wherein the sensor device further comprises:
a sensor device power source configured to provide power to the sensor device.

12. The article in accordance with claim 11, wherein the wristband device further comprises:
a wristband device power source configured to provide power to the wristband device.

13. The article in accordance with claim 12, further comprising:
a power source interface configured to provide electrical contact between the sensor device and the wristband device power source, wherein the sensor device is configured to receive power from at least one of the wristband device power source via the power source interface and the sensor device power source.

14. The article in accordance with claim 10, wherein the wristband device further comprises:
a wristband device power source configured to provide power to the wristband device.

15. The article in accordance with claim 14, further comprising:
a power source interface configured to provide electrical contact between the sensor device and the wristband device power source, wherein the sensor device is configured to receive power from the wristband device power source via the power source interface.

16. The article in accordance with claim 14, wherein the wristband device power source comprises a battery.

17. The article in accordance with claim 7, wherein the wristband device comprises a bracelet.

18. The article in accordance with claim 17, wherein the removable portion comprises a bracelet link of the bracelet, and wherein the sensor device is disposed within the bracelet link of the bracelet.

19. The article in accordance with claim 7, wherein the communication logic comprises a wireless interface for wirelessly coupling the sensor device to the external electronic device.

20. The article in accordance with claim 19, wherein the wireless interface is a short range wireless interface.
